(19) United States
(12) Patent Application Publication
(10) Pub. No.: US 2016/0030257 A1
(43) Pub. Date: Feb. 4, 2016

(54) ABSORBENT SANITARY ARTICLE AND MACHINE FOR MAKING THE ARTICLE

(71) Applicant: GDM S.P.A., Bologna (IT)

(72) Inventors: Matteo PIANTONI, Albino (Bergamo) (IT); Valerio SOLI, Bologna (IT)

(21) Appl. No.: 14/781,512

(22) PCT Filed: Apr. 17, 2014
(86) PCT No.: PCT/IB2014/060804

§ 371 Date: Sep. 30, 2015

(30) Foreign Application Priority Data
Apr. 18, 2013 (IT) BO2013A000177

(51) Int. Cl. A61F 13/538; A61F 13/15; A61F 13/539

(52) U.S. Cl. CPC A61F 13/538; A61F 13/539; A61F 13/15764; A61F 13/15634; A61F 2013/530569

(57) ABSTRACT

An absorbent sanitary article that includes a permeable inner layer, an impermeable outer layer, an absorbent pad interposed between the permeable inner layer and the impermeable outer layer; the pad includes a first layer of non-woven fabric, a second layer of non-woven fabric partly joined to the first layer of non-woven fabric, an absorbent core interposed between the first and second layers of non-woven fabric, a plurality of seals between the first layer of non-woven fabric and the second layer of non-woven fabric defining in the absorbent pad a plurality of cells; the absorbent core includes a first absorbent material and a second absorbent material divided in the cells; the cells comprise at least a first cell containing the second absorbent material together with the first absorbent material.

### TECHNICAL FIELD

This invention relates to an absorbent sanitary article, such as a baby nappy or the like, and to a machine for making articles of this kind.

In this description, for simplicity, express reference is made to a nappy but without thereby limiting the scope of the invention.

### BACKGROUND ART

As is known, nappies comprise an absorbent pad or padding which is normally enclosed between a permeable inner layer of non-woven fabric and an impermeable outer layer of polyethylene.

Absorbent pads of known type comprise an absorbent core made of an absorbent material, such as, for example, granules of superabsorbent polymer material (SAP) inside a mixture of containment cellulose pulp (fluff) and absorbent material binder, sandwiched between two layers of non-woven fabric.

In order to improve drainage of the absorbed liquid and, at the same time, reduce production costs, manufacturers are opting for absorbent cores with more and more granular superabsorbent material and less and less cellulose pulp, and even absorbent cores that are totally free of cellulose.

One example of absorbent pads comprising absorbent cores made of superabsorbent polymer material and substantially free of cellulose is described in document EP2444046.

The padding described in that prior art document comprises a first layer of non-woven fabric, a second layer of non-woven fabric and the cellulose free absorbent material enclosed between the two layers of non-woven fabric.

The two layers of non-woven fabric are joined by seals which are suitably made in such a way as to form a plurality of cells containing the absorbent material which is distributed uniformly in all the cells.

In one embodiment, the seals which separate the cells are uninterrupted so as to better contain and position the absorbent material, especially when the product is new, unused and the superabsorbent material dry.

In a second embodiment, the seals have suitable breaks in them so as to improve the diffusion of the liquid and optimize its absorption by the absorbent material.

Generally speaking, the seals provide preferential channels through which the liquid spreads within the nappy. However, based on the quantity of the liquid and on the speed at which it spreads, the absorbent material may expand in a non-uniform and non-optimal manner, thus altering its optimal distribution within the pad and reducing the effectiveness of the nappy itself.

### AIM OF THE INVENTION

In this context, the main technical purpose of this invention is to propose an absorbent sanitary article which is free of the above mentioned disadvantages.

The aim of this invention is to propose an absorbent sanitary article whose pad is substantially cellulose free and whose absorption capacity is optimized, irrespective of the quantity of liquid and the flow thereof.

The technical purpose and aims specified are substantially achieved by an absorbent sanitary article according to claim 1 and by a machine according to claim 10 for making the absorbent sanitary article.

### BRIEF DESCRIPTION OF THE DRAWINGS

Further features and advantages of this invention are more apparent in the description below, with reference to preferred, non-exclusive embodiments of an absorbent sanitary article and of a machine for making the absorbent sanitary article, as illustrated in the accompanying drawings, in which:

FIG. 1 is a schematic plan view, with some parts cut away for better clarity, illustrating an absorbent sanitary article according to this invention;

FIG. 2 is a schematic cross section of the article of FIG. 1;

FIG. 3 is a schematic front view, with some parts cut away for better clarity, illustrating a machine for making the absorbent article of the preceding figures.

### DETAILED DESCRIPTION OF PREFERRED EMBODIMENTS OF THE INVENTION

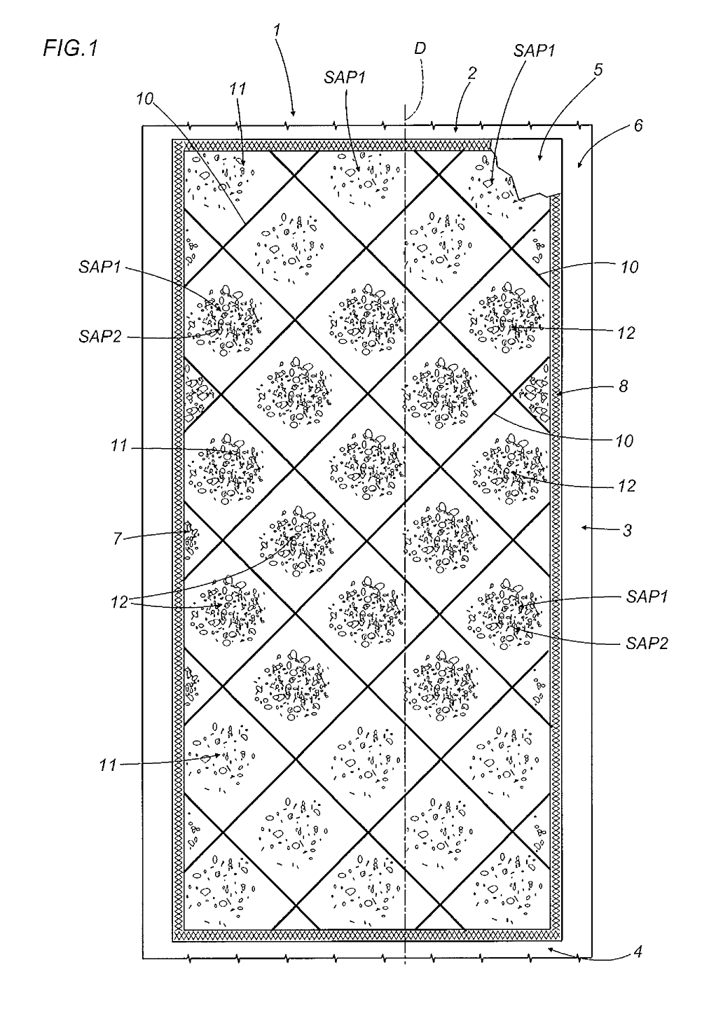

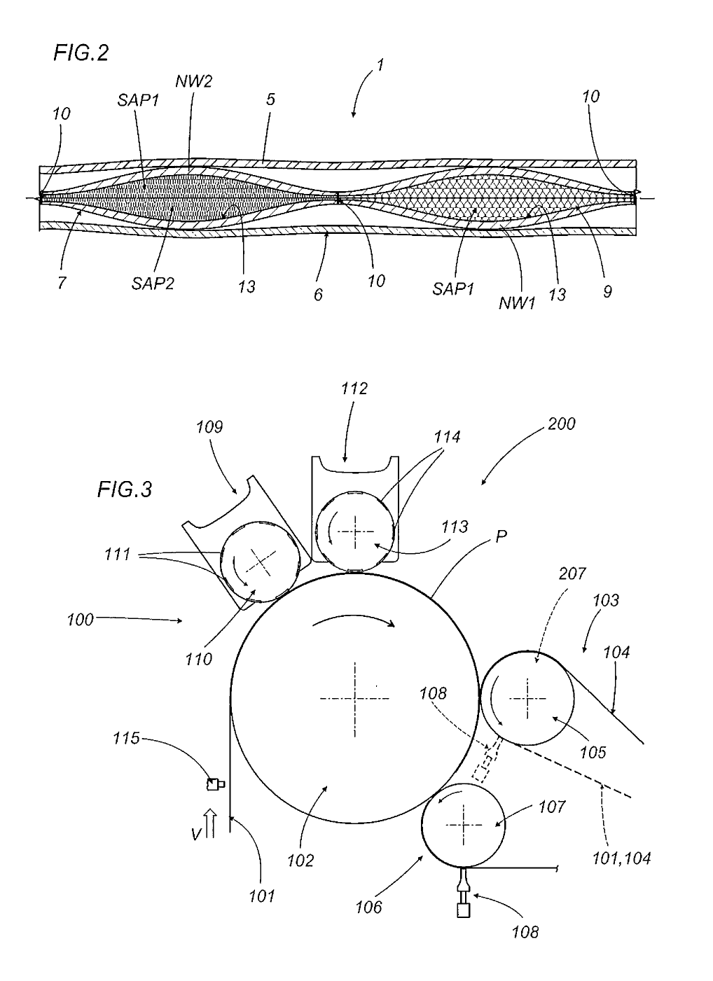

With reference in particular to FIGS. 1 and 2, the numeral 1 denotes an absorbent sanitary article according to the invention, only partly illustrated, such as for example, a baby nappy, to which express reference is hereinafter made without thereby limiting the scope of the invention, sanitary towels or the like.

The absorbent sanitary article 1 extends along a main longitudinal direction of extension D and has a front zone 2, a central zone 3 and a rear zone 4.

In a preferred embodiment, the main extension is the longitudinal one, that is the extension measured between the front zone and the rear zone of the article 1.

Preferably, the front zone 2 has a length, measured along the main direction of extension D, which is smaller than the length of the rear zone 4, measured along the same main direction of extension D.

The article 1 comprises an inner layer 5 permeable to liquids, also known as “top sheet”, preferably defined by a piece of non-woven fabric, and an outer layer 6 impermeable to liquids, also known as “back sheet”, preferably defined by a sheet of polyethylene.

The top sheet and the back sheet are joined to each other in known manner to define the outer container of the article 1.

The article 1 comprises an absorbent pad 7 interposed between the permeable inner layer 2 and the impermeable outer layer 3 and delimited by an outer perimeter 8.

Considering the longitudinal extension of the article 1, the pad 7 is positioned in the central zone 3 of the article.

In practice, the pad 7 defines the central zone and delimits the front zone 2 and the rear zone 4.

The pad 7 comprises a first layer NW1 of non-woven fabric and a second layer NW2 of non-woven fabric at least partly joined to the first layer, as explained in more detail below.

The pad 7 comprises a core 9 interposed between the first layer NW1 and the second layer NW2.

The pad 7 has a plurality of seals 10 between the layer NW1 of non-woven fabric and the layer NW2 of non-woven fabric.

The seals 10 define in the pad 7 a plurality of cells 11 for containing the core 9 which is, in practice, divided in the cells 11.

In one embodiment, the seals 10 comprise a peripheral seal 10a which extends at least partly round the perimeter 8 of the pad 7.

In an embodiment not illustrated, the core 9 comprises a first absorbent material SAP1, preferably a polymer, to which express reference is made herein without thereby limiting the invention and which is located in certain cells 11.

The core 9 comprises a second absorbent material SAP2, preferably a polymer, to which express reference is made herein without thereby limiting the invention and which is located in other cells 12.

Thus, the pad 7 comprises cells 11 containing the first material SAP1 and cells 12 containing the second absorbent polymeric material SAP2.

In the preferred embodiment illustrated, the core 9 comprises a first absorbent material SAP1, preferably a polymer, to which express reference is made herein without thereby limiting the invention and which is divided in the cells 11 which, in other words, each enclose a quantity of the first absorbent polymeric material SAP1.

The core 9 comprises a second absorbent material SAP2, preferably a polymer, to which express reference is made herein without thereby limiting the invention and which is divided in the cells 11.

Preferably, the pad 7 has a plurality of cells 12 containing the second absorbent polymeric material SAP2 together with the absorbent polymeric material SAP1.

The pad 7 is divided into cells 11, some of which—the cells 12—contain both the first and the second absorbent polymeric material SAP1 and SAP2.

In one embodiment, the first absorbent polymer material SAP1 and the second absorbent polymer material SAP2 are the same absorbent polymer material.

In that case, the cells 12 contain a larger quantity of absorbent polymeric material than the other cells 11.

Advantageously, the cells 12 containing more absorbent polymeric material have a higher absorption capacity than the other cells 11 which have less absorbent polymeric material in them.

In one embodiment, the second absorbent polymer material SAP2 is different from the first absorbent polymer material.

In that case, the cells 12 contain a predetermined quantity of the first absorbent polymeric material and a predetermined quantity of the second absorbent polymeric material.

Preferably, the response time to liquid of the second absorbent polymeric material SAP2 is longer than the response time to liquid of the first absorbent polymeric material SAP1.

As is known, the response time to liquid is a specific property of the superabsorbent material.

The response time to liquid may, for example, be a function of the permeability and/or liquid retaining capacity and/or absorption and/or retention and/or absorption against pressure of the superabsorbent polymer material. In short, the performance of superabsorbent polymer materials is determined mainly, in substantially known manner, by: permeability coefficient, apparent specific gravity (bulk density), flow rate, centrifuge retention capacity (CRC) and particle size distribution (PSD).

These properties are preferably determined and identified according to the Worldwide Strategic Partners (WSP) test methods.

In other words, the first material SAP1 starts absorbing liquid before the second material SAP2 which starts absorbing the liquid at a later stage. That way, during the initial stages of absorption, the second absorbent material SAP2 is permeable to the liquid which is thus free to reach the first absorbent material SAP1.

The second absorbent polymeric material SAP2 starts absorbing after the first material SAP1, thus guaranteeing more continuity in the performance of the article 1.

In one embodiment, the cells 12 contain the same quantity of absorbent material SAP1 and material SAP2.

Advantageously, the cells 12 in the pad 7 are located more towards the front zone 2, that is to say, the cells 12 are closer to the front zone 2 than they are to the rear zone 4.

In the pad 7, the first cells 12 are offset from a median section transversal to the direction D.

The cells 12 with higher absorbing power are located, preferably, in the part of the nappy most affected by the flow of liquid discharged by the wearer, not illustrated.

This configuration allows keeping the performance of the article 1 at a high level and, if necessary, saving absorbent material at the end portions of the pad 7.

In one embodiment, the pad 7 comprises an adhesive layer 13 between the layer NW1 and the absorbent polymer material SAP1 and SAP2.

The adhesive layer 13 mainly has a process function, as will become clearer as this description continues, that is to say, it serves to keep the absorbent material in position while the pad 7 is being formed.

With reference to FIG. 3, the numeral 200 denotes in its entirety a machine for making absorbent sanitary articles 1.

This description of the machine 200 is limited to the parts necessary for understanding this invention.

The machine 200 comprises a feed line, of substantially known type and not illustrated, for feeding at least one continuous web of impermeable material used to make the impermeable outer layer 6.

The machine 200 comprises a forming unit 100 for making the absorbent pads 7 to form a succession of the pads 7 and to associate them with the continuous web of impermeable material.

The unit 100 comprises a system for feeding a first web 101 of non-woven fabric in a feed direction V along a path P for defining the first layer NW1) of non-woven fabric.

The feed system comprises a suction roller 102 for holding down and feeding at least the first web 101 of non-woven fabric and rotatable in a clockwise direction.

The roller or drum 102 uses suction to hold down the web 101 and, preferably, also the further layers of the article 1 subsequently placed on the web 101, as will become clearer as this description continues.

The unit 100 comprises a station 103 for placing a second web 104 of non-woven fabric on the first web 101 of non-woven fabric to define the second layer NW2 of non-woven fabric of the pad 7.

The station 103 comprises a roller 105 for transferring the second web 104 and tangent to the suction roller 102.

The roller 105 is preferably rotatable in anticlockwise direction.

The unit 100 comprises a sealing station 106 by which the second web 104 is joined to the first web 101 so as to close the layers NW1 and NW2 on the cores 9 of the pads 7.

The sealing station 106 comprises a contact roller 107, for example rotatable in anticlockwise direction, tangent to the suction roller 102 and located downstream of the transfer roller 105 relative to the feed direction V of the absorbent pads 7.

The roller 107 has on its outside surface a pattern of the seals 10, 10a.

The sealing station 106 comprises a sonotrode 108, of substantially known type, for making the seals 10, 10a and facing the outside surface of the roller 107.

The sonotrode 108 acts on the contact roller 107 and, more specifically, makes the seals 10, 10a based on a predetermined pattern.

In one embodiment, shown by dashed lines, the roller 105 itself defines the contact roller 107 and has the sealing pattern formed on its cylindrical outside surface.

In that case, the sonotrode 108 acts on the outside surface of the roller 105 to make the seals 10.

In an alternative embodiment not illustrated, the sealing station 106 comprises the suction roller 102 which defines the contact roller 107.

The pattern of the seals 10 is formed on the cylindrical outside surface of the roller 102 and the sonotrode 108 acts on the roller 102.

Preferably, the sonotrode 108 is in the form of a pair of rotary sonotrodes.

The unit 100 comprises a first station 109 for laying out the absorbent polymer material SAP1 on the web 101 of non-woven fabric.

The station 109 is operatively positioned upstream of the placing station 103 along the feed direction V.

The station 109 comprises a laying-out roller 110 which is rotatable, for example in anticlockwise direction, and which is substantially tangent to the suction roller 102.

The roller 110 is provided, on its outside cylindrical surface, with a plurality of pockets 111, each feeding and placing on the web 101 along the path P the first absorbent material SAP1.

The pockets 111 are preferably located on the outside surface of the first roller as a function of the position of the cells 11 in the pad 7.

The forming unit 100 comprises a second station 112 for laying out the second absorbent polymeric material SAP2 and located downstream of the first laying-out station 109 and upstream of the placing station 106 along the feed direction V.

As illustrated, the second laying-out station 112 comprises a laying-out roller 113 substantially tangent to the suction roller 102 and rotatable, for example, in anticlockwise direction.

The roller 113 is provided, on its outside cylindrical surface, with a plurality of pockets 114, each feeding and placing on the web 101 along the path P a portion of the second absorbent material SAP2.

The pockets 114 are located on the outside surface of the roller 113 as a function of the position of the cells 12 in the pad 7.

The forming unit 100 comprises an adhesive dispenser 115 operating on the web 101 upstream of the laying-out station 109 to apply the adhesive layer 13 on the web 101 of non-woven fabric.

The machine 200 comprises a line, of substantially known type and not illustrated, for feeding and applying at least one continuous web of permeable material used to make the permeable inner layer 5.

The pads 7 are interposed between the outer layer 6 and the inner layer 5.

1. An absorbent sanitary article extending along a main longitudinal direction of extension, having a front zone, a central zone and a rear zone, and comprising
a permeable inner layer,
an impermeable outer layer,
an absorbent pad interposed between the permeable inner layer and the impermeable outer layer, and comprising
a first layer of non-woven fabric
a second layer of non-woven fabric partly joined to the first layer of non-woven fabric
an absorbent core interposed between the first and second layers of non-woven fabric and comprising a first absorbent material, the absorbent pad comprising
a plurality of seals between the first layer of non-woven fabric and the second layer of non-woven fabric defining in the absorbent pad a plurality of cells,
the first absorbent material being divided in the cells, the absorbent sanitary article being characterized in that the absorbent core comprises a second absorbent material and the cells comprise at least a first cell containing the second absorbent material.

2. The absorbent sanitary article according to claim 1, wherein the first cell contains the second absorbent material together with the first absorbent material.

3. The absorbent sanitary article according to claim 1, wherein the absorbent pad comprises a plurality of the first cells located further towards the front zone of the absorbent sanitary article.

4. The absorbent sanitary article according to claim 1, wherein the first cells in the absorbent pad are offset relative to a transverse mid-section.

5. The absorbent sanitary article according to claim 1, wherein the second absorbent material is different from the first absorbent material.

6. The absorbent sanitary article according to claim 5, wherein the second absorbent material has a longer response time to liquid than the first absorbent material.

7. The absorbent sanitary article according to claim 1, wherein the first cell comprises the same quantity of the first absorbent material and of the second absorbent material.

8. The absorbent sanitary article according to claim 1, wherein the absorbent pad comprises an adhesive layer between the first layer of non-woven fabric and the first and second absorbent materials.

9. The absorbent sanitary article according to claim 1, wherein the absorbent pad has an outer perimeter and comprises a peripheral seal joining at least the first and second layers, of non-woven fabric, the peripheral seal extending at least partly along the outer perimeter.

10. A machine for making absorbent sanitary articles according to claim 1, the machine comprising a line for feeding at least one continuous web of impermeable material for defining the impermeable outer layer, a line for feeding at least one continuous web of permeable material for defining the permeable inner layer and at least one unit for forming the absorbent pads to form a succession of the absorbent pads and to associate the absorbent pads with the continuous web of impermeable material, the forming unit comprising
a system for feeding a first web of non-woven fabric in a feed direction along a path for defining the first layer of non-woven fabric,
a station for placing a second web of non-woven fabric on the first web of non-woven fabric for defining the second layer of non-woven fabric,
a station for sealing the second web of non-woven fabric to the first web of non-woven fabric,
a first station for laying out the first absorbent material on the first web of non-woven fabric and being operatively positioned upstream of the first placing station relative to the feed direction, the machine wherein that the forming unit comprises a second station for laying out the second absorbent material and being positioned downstream of the first laying-out station and upstream of the sealing station.

11. The machine according to claim 10, wherein the forming unit comprises an adhesive dispenser operating at the feed system to spread the adhesive layer on the first web of non-woven fabric.

12. The machine according to claim 10, wherein the feed system comprises a suction roller for holding down and feeding at least the first web of non-woven fabric.

13. The machine according to claim 12, wherein the first laying-out station and the second laying-out station comprise, respectively, a first laying-out roller, substantially tangent to the suction roller, and a second laying-out roller, substantially tangent to the suction roller, the first laying-out roller having on its cylindrical outer surface a plurality of first pockets each feeding and placing along the path a portion of the first absorbent material, the first pockets being located on the outer surface of the first laying-out roller according to the position of the cells in the absorbent pad,
the second laying-out roller having on its cylindrical outer surface a plurality of second pockets each feeding and placing along the path a portion of the second absorbent material, the second pockets being located on the outer surface of the second laying-out roller according to the position of the first cells in the absorbent pad.
